(19) United States
(12) Patent Application Publication
(10) Pub. No.: US 2026/0140299 A1
(43) Pub. Date: May 21, 2026

(54) METHOD FOR MANUFACTURING TRANSMITTER DEVICE AND TRANSMITTER DEVICE

(71) Applicants: TOHOKU UNIVERSITY, Sendai-shi (JP); The University of Osaka, Osaka (JP)

(72) Inventors: Yoichi HAGA, Sendai-shi (JP); Noriko TSURUOKA, Sendai-shi (JP); Kodai TAKEYA, Sendai-shi (JP); Yoshiaki SAKAI, Sendai-shi (JP); Makoto OSANAI, Osaka (JP)

(73) Assignees: TOHOKU UNIVERSITY, Sendai-shi (JP); The University of Osaka, Osaka (JP)

(21) Appl. No.: 19/389,543

(22) Filed: Nov. 14, 2025

(30) Foreign Application Priority Data
Nov. 15, 2024 (JP) 2024-200146

(51) Int. Cl. F21V 8/00; G02B 6/42

(52) U.S. Cl. CPC G02B 6/0065; G02B 6/0028; G02B 6/4281

(57) ABSTRACT

A method for manufacturing a transmitter device includes: forming a transmitter on a member in a form of a column shape or deformed into a column shape; providing a light guide to an end portion of the member; and carrying out a process on an end face of the member to expose the transmitter and the light guide such that the transmitter and the light guide are flush with the end face of the member.

### CROSS-REFERENCE TO RELATED APPLICATION

This application is based upon and claims the benefit of priority of the prior Japanese Patent application No. 2024-200146, filed on Nov. 15, 2024, the entire contents of which are incorporated herein by reference.

### FIELD

The present disclosure relates to a method of manufacturing a transmitter device and a transmitter device.

### BACKGROUND

Activity of nerve cells in the brain is accomplished by an electric pulse signal called an action potential transmitted along an axon from a cell body. Action potentials can be provoked by external light or electrical stimulation. A neural electrode is a device for measuring activity information of a nerve cell and inputting information to a nervous system by inserting an electrode needle into a soft biological tissue such as a brain or a nerve bundle in order to examine the activity of the cell. Furthermore, some neural electrodes have been developed which can be used in combination with an optical fiber or an endoscope (Patent Literature 1 and Patent Literature 2).

Patent Literature 1 discloses a neural electrode using a flexible board. Flexible boards have used in devices in various fields (Non-Patent Literature 1). Patent Literature 2 discloses a living body tube including a lens at a tip end thereof.

### RELATED ART DOCUMENTS

### Patent Document

[Patent Literature 1] Japanese Laid-open Patent Publication No. 2023-71354
[Patent Literature 2] Japanese U.S. Pat. No. 7,489,072
[Non-Patent Document 1] “3 D Self-Assembled Microelectronic Devices: Concepts, Materials, Applications”, Daniil Karanaushenko et al., Advanced Materials, 2020, 32, 1902994

### SUMMARY

The method for manufacturing a transmitter device, which is disclosed herein, includes: forming a transmitter on a member in a form of a column shape or deformed into a column shape; providing a light guide to an end portion of the member; and carrying out a process on an end face of the member to expose the transmitter and the light guide such that the transmitter and the light guide are flush with the end face of the member.

### BRIEF DESCRIPTION OF DRAWINGS

FIG. 1 is a schematic diagram illustrating a configuration of a transmitter device formed of a flexible board according to a first embodiment;

FIG. 2 is a schematic diagram illustrating a flexible board (Flexible Printed Circuit (FPC));

FIG. 3 is a schematic diagram illustrating a front end face;

FIG. 4 is a diagram illustrating a process of manufacturing a transmitter device of the first embodiment;

FIG. 5 is a diagram illustrating a reshaping process of reshaping a flexible board;

FIG. 6 is a diagram illustrating a gluing process of gluing a flexible board to a supporting body;

FIGS. 7A-7C are diagrams each schematically illustrating a transmitter device formed by wrapping flexible board multiple times according to a modification;

FIG. 8 is a schematic diagram illustrating a configuration of a transmitter device formed of a supporting body according to a second embodiment;

FIG. 9 is a schematic diagram illustrating a wiring pattern formed on the surface of the supporting body;

FIG. 10 is a schematic diagram illustrating a front end face;

FIG. 11 is a diagram illustrating a process of manufacturing a transmitter device of the second embodiment;

FIGS. 12A and 12B are schematic diagrams each illustrating a configuration including an optical waveguide in place of a wire;

FIGS. 13A and 13B are schematic diagrams each illustrating a configuration in which a mirror is provided to an optical waveguide;

FIG. 14 is a diagram illustrating an endoscope apparatus with a transmitter device;

FIG. 15 is a diagram illustrating an optical fiber apparatus with a transmitter device; and

FIGS. 16A and 16B are diagrams illustrating GRIN lenses having respective different pitches.

### DESCRIPTION OF EMBODIMENT(S)

In recent years, attempts have been made to elucidate the activity of nerve cells in the deep brain by using a neural electrode in combination with an endoscope or an optical fiber. In a configuration in which an electric measuring unit is arranged on a long axis face of a tube of the neural electrode and is not located on the same face as an observation unit of an endoscope and an optical fiber, a brain site being measured is different from a brain site being observed. For the above, a demand arises for a neural electrode (transmitter device) that can measure a site being observed.

Description will now be made in relation to a transmitter device and a method for manufacturing a transmitter device according to embodiments with reference to the accompanying drawings. Furthermore, referring to the drawings, description will be made in relation to an endoscopic apparatus with a transmitter device and an optical fiber apparatus with a transmitter device as application examples of the transmitter device. Specifically, the transmitter device of the present embodiments means an electric conductor and an optical waveguide. The following embodiment is merely illustrative and is not intended to exclude the application of various modifications and techniques not explicitly described in the embodiment. Each configuration of the present embodiments can be variously modified and implemented without departing from the scopes thereof. Also, the configuration can be selected or omitted according to the requirement or appropriately combined.

In the following description of the transmitter device, on the basis of a state where a transmitter device is placed on a horizontal face, an end provided with an electrode (recording electrode) or an optical waveguide (exit of the optical waveguide) is defined as the front, the opposite of the front is defined as the rear, and the left and right are defined on the basis of the front-rear. The up-down direction is defined on the basis of the direction of gravity being defined as a downward direction and the reverse direction thereof being defined as an upward direction. In addition, a direction toward the center of the vertical cross section (longitudinal section) along the front-rear direction of the transmitter device is defined as an inner side (inside), and a direction toward outer circumference of the longitudinal section is defined as an outer side (outside).

### I. Premise

The transmitter device of the present disclosure includes a conductor and/or an optical waveguide and aims at acquiring a signal from a measurement target. The transmitter device including an electrode can observe and measure the electric property of an observation site to be observed of the measuring target by bringing the electrode close to or into contact with the observation site. In addition, the transmitter device including the optical waveguide can observe and measure the optical property of an observation site to be observed of the measuring target by irradiating the observation site with light. This means that the transmitter device of the present disclosure is a measuring device or an instrumentation device.

The transmitter device of the present embodiment may include one of a conductor and an optical waveguide or the both. If including both a conductor and an optical waveguide, the transmitter device can measure and observe both the electrical and optical properties of the observation site. In the present embodiments, description will be made in relation to a neural electrode, which measures the electric potential of the brain and is regarded as a lower concept of the transmitter device.

Here, the description assumes that the neural electrode can be used in combination with a separate insert exemplified by an endoscope or an optical fiber. The neural electrode according to the embodiments is for measuring the basal ganglia of the cerebrum, which is located deep in the brain and is involved in the start and stop of motion. For this purpose, the neural electrode is placed on the surface of the brain or inserted into a groove of the brain. This usage requires to satisfy the following requirements.
(a) Ensuring a Bore of 350-550 μm
(b) six electrodes arranged equidistantly (at inter-electrode angle of 60 degrees) on the circumference of the circle
(c) possible minimum outer diameter for the purpose of minimal invasion
(d) rigidity that can withstand penetration into the brain
(e) biocompatibility

### II. Embodiment

Hereinafter, description will be made in relation to a transmitter device formed of a member deformed into a column shape and a method for manufacturing such transmitter device in Item (A) as a first embodiment, and a transmitter device formed of a member into a column shape and a method for manufacturing such transmitter device in Item (B) as a second embodiment. Further description will be made in relation to a transmitter device provided with an optical waveguide as a third embodiment in Item (C). Then, application examples of a transmitter device will be described in Item (D).

### A. First Embodiment

### 1. Configuration

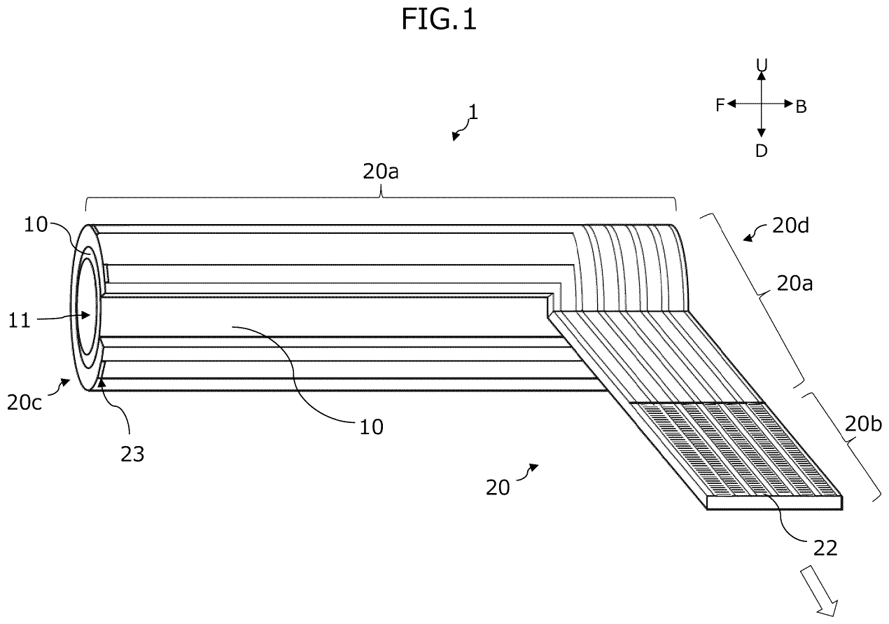

Description will now be made in relation to a transmitter device 1 including conductors and being formed of a flexible board according to a first embodiment with reference to FIGS. 1-5. FIG. 1 is a schematic diagram illustrating a configuration of the transmitter device 1 formed of a flexible board 20 according to the first embodiment. The transmitter device 1 according to the present embodiment is formed of a flexible board 20, which is a member deformed into a column shape, and includes a recording electrode 23 provided at an end portion of the flexible board 20. The recording electrodes 23 are formed on an end face 20c of the flexible board 20.

In the transmitter device 1 of the present embodiment, the flexible board 20 is provided on a column-shaped supporting body 10. The supporting body 10 is a skeletal member that supports a pressure load acting on the transmitter device 1. The supporting body 10 has a column shape which is exemplified by a cylindrical or prism shape. The vertical cross-sectional shape of the supporting body 10 along the front-rear direction is formed into, for example, a circle or an ellipse, but is not limited thereto. The outer diameter of the cross section and the length in the front-rear direction of the supporting body 10 have sizes that allows a user to grip the transmitter device 1 by hand when the user uses the transmitter device 1. The inner diameter of the cross section of the supporting body 10 has a size that allows inserts 40 (e.g., a lens 41, an endoscope 42, and/or an optical fiber 43), which will be described below, to be inserted. The supporting body 10 according to the present embodiment has an elongated cylindrical shape. One of the examples of the material for a neural electrode is a ceramic tube made of zirconia (Kyocera Co., Ltd., outer diameter of 0.8 mm, inner diameter 0.55 mm) that satisfies the above-described requirements (d) and (e).

A through-path 11 formed in the supporting body 10 extends from a first end and a second end along the front-rear direction and can contain the insert 40 therein. The through-path 11 also functions as a flow path for collecting a tissue fluid from living tissue and injecting a chemical fluid into living tissue.

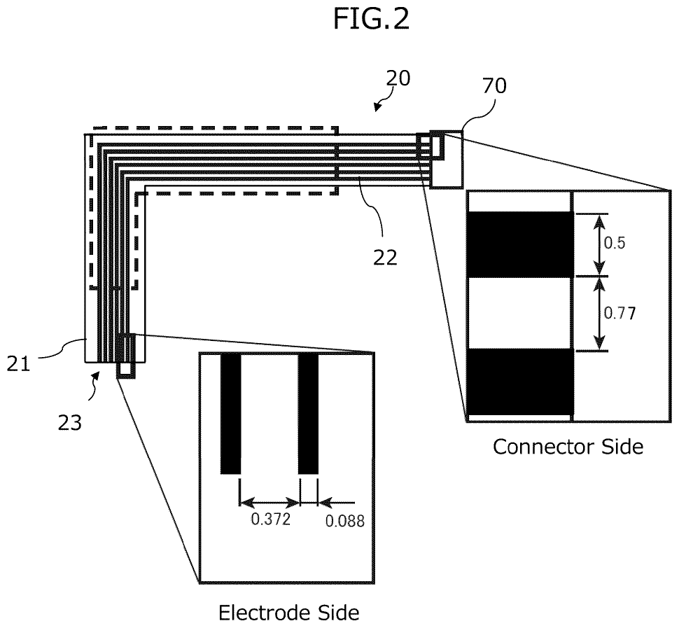

FIG. 2 is a schematic view illustrating the flexible board 20. A flexible board (hereinafter, also referred to as a FPC (Flexible Printed Circuit)) 20 is an electrical circuit for measuring an electrical signal of biological tissue and also for electrically stimulating biological tissue.

The FPC 20, which is a planar-shaped member, is deformed into a column shape. For this purpose, the FPC 20 is formed of a thermoplastic material, such as a Liquid Crystal Polymer (LCP) sheet, which can be deformed and keep its shape by being heated. LCP is excellent in thermoplastic properties and can reduce resilient force. One of the examples of an LCP sheet for a neural electrode is a liquid crystal polymer (Felios LCP, Panasonic, LCP thickness of 25 μm, copper foil thickness of 9 μm) including a copper foil and having a thermoplastic property. Incidentally, a suitable material for a structure in which the FCP 20 is wrapped around the supporting body 10 once (singly ply) is LCP and a suitable material for a structure in which the FCP 20 is wrapped multiple times (multiple plies) is a polyimide sheet (12.5 μm, Capton, Toray DuPont Co.), which can be easily thinned.

The FPC 20 is in a rectangular shape or in shape of a combination of rectangular shapes. As illustrated in FIG. 1, a recording electrode 23 is provided on a first end of the FPC 20 and a second end of the FPC 20 is connected to a connector 70. On the top face (top surface) of the FPC 20, conductor (hereinafter also referred to as conductor tracks or wires) 22 that connect a first end portion with a recording electrode (hereinafter also referred to as an electrode) 23 to a second end portion connected to the connector 70 continuously extends.

The wiring width of a connector portion 20b illustrated in FIG. 1 is designed to match the width of a pin of the connector, and the length in the left-right direction of the connector portion 20b preferably has a sufficient length exemplified by a length 1.5 times longer than the minimum length for taking out the conductors 22. At the tip of the connector part 20b, a portion to which all the wires are connected is provided to facilitate the connection during electroplating. In the present embodiment, the recording electrodes 23 are exposed on the end face of the FPC 20 by cutting a part surrounded by a dashed line of the FPC 20 where the conductors 22 are formed in FIG. 2.

The FPC 20 of the present embodiment is provided with six conductors 22, which may each have a constant width or, as illustrated in FIG. 2, may each have a width that widens from the recording electrode 23 to the connector portion 20b, in contrast, narrow the interval (wiring interval, distance between the wires) of each consecutive wires from the recording electrode 23 to the connector portion 20b. The wiring interval may be uniform entirely from the first end to the second end of the FPC 20. In the example of FIG. 2, each conductor 22 has a width of 0.088 mm on the electrode side, a width of 0.5 mm on the connector side and a wiring interval of 0.372 mm on the electrode side, and a wiring interval of 0.77 mm on the connector side.

As illustrated in FIG. 1, the FPC 20 of the present embodiment includes an adhesion portion 20a having a lower face (bottom face) part of which adheres to the supporting body 10 and the connector portion 20b having a lower face not adhering to the supporting body 10. The connector portion 20b extends from a part (e.g., a front end portion or rear end portion) of the adhesion portion 20a, which extends in the front-rear direction, in a direction deviating from the outer circumference face. The FPC 20 of the present embodiment extends tangentially to the outer circumference face of the supporting body 10, and is formed into a shape of point symmetry of a so-called L-shape (i.e., a shape formed by combining rectangles).

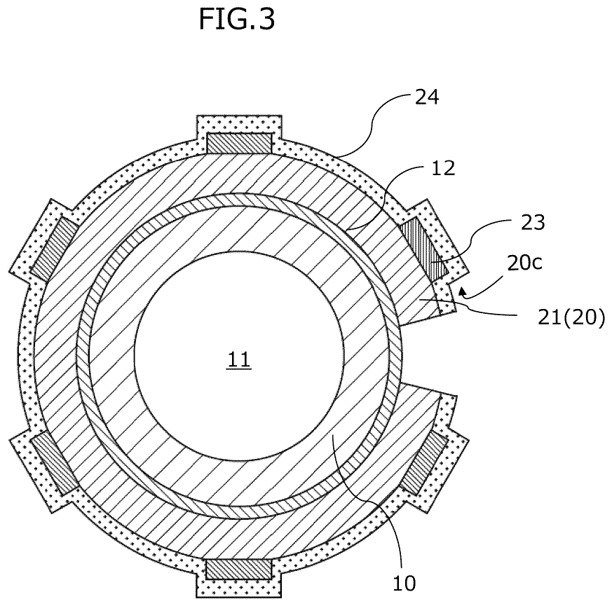

The adhesive portion 20a is wrapped around the outer circumference face of the supporting body 10 and is deformed into a column shape by an adhesive 12 (see FIG. 3) applied between the supporting body 10 and the adhesive portion 20a to adhere to the supporting body 10. Preferably, the wiring interval of the conductors 22 is preferably designed such that the conductors 22 are arranged at regular intervals when the thickness of the adhesive 12 applied to the adhering portion of the adhesion portion 20a is set to a predetermined value (e.g., 15 μm). In one of the examples of a neural electrode, the width and the thickness of wiring may be designed to satisfy the electrode area of 707 μm², considering side etching in the Cu etching. The manner of adhering will be described below.

In addition, if the transmitter device 1 is used by being inserted into a measuring target, the adhesion portion 20a preferably has a sufficient front-rear length considering a length in the insertion direction of the connector portion 20b (i.e., the entire length in the front-rear direction of the connector portion 20b), and the front-rear length of the adhesion portion 20a is preferably longer than one time the insertion length, for example. As an example of a neural electrode, the adhesive 20a has an sufficient insertion length of 17.15 mm, which is calculated by subtracting the length in the insertion direction (in the front-rear direction) of the connector portion 20b from the total length 25 mm in the front-rear direction of the adhesion portion 20a with respect to a minimum insertion length of 10 mm.

As shown in FIG. 1, the flexible board 20 deformed or formed into a column shape includes a through-path 11 that penetrates between an electrode-side end face 20c on which the recording electrode 23 is formed and a connector-side end face 20d on the second end.

FIG. 3 is a schematic diagram illustrating a front end face of the transmitter device 1. In this drawing, the illustration of the connector portion 20b is omitted. The front end face includes, sequentially in the outward direction from the through-path 11, the supporting body 10, the adhesive 12, an LCP 21 (one type of the FPC 20) adhering to the outer circumference face of the supporting body 10 via the adhesive 12, the recording electrodes 23 formed on the electrode-side end faces 20c of the conductors 22, and an insulating layer 24 that covers the conductors 22. The recording electrodes 23 may be provided at equal intervals, and the angle between the electrodes may be 60 degrees.

As illustrated in FIG. 3, a part not covered with FPC 20 extends over the supporting body 10, but this is merely a minute gap generated due to designing. By making the length of the outer circumference of the supporting body 10 the same as the length in the left-right direction of the adhesion portion 20a, the transmitter device 1 is formed not to have such a gap.

At the end-face side portion (front end face of the transmitter device 1) of the through-path 11, a light guide made flush with the end face may be provided. Hereinafter, description assumes that the light guide is a lens, but the light guide may alternatively by an optical fiber 43 in place of a lens 41. An example of the lens 41 is a GRadient INdex (GRIN) lens (which may also be referred to as a GRIN rod lens). The GRIN lens 41 will be described in an applied example in Item “C”.

### 2. Method for Manufacturing

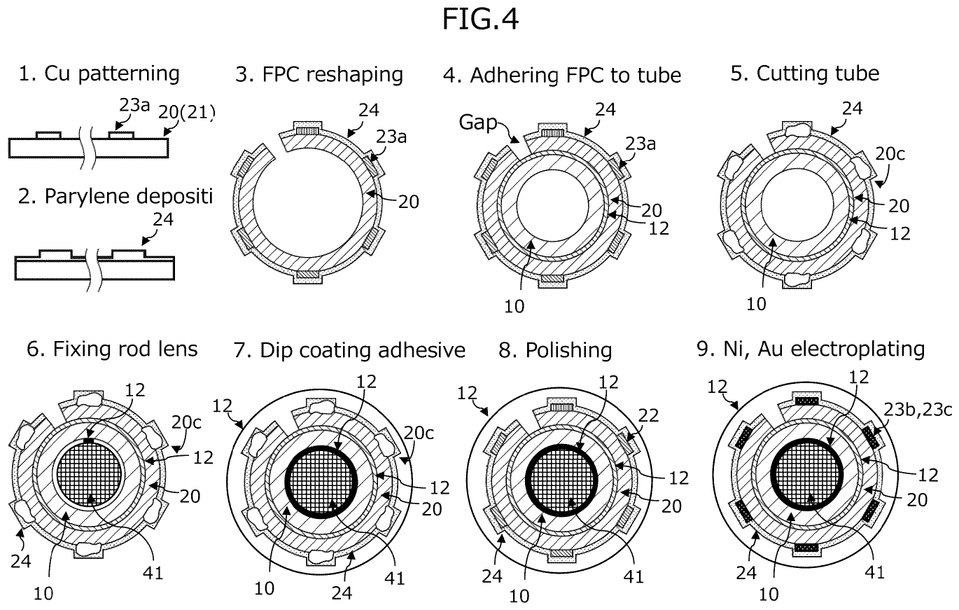

Hereinafter, description will now be made in relation to a method of manufacturing the transmitter device 1 with reference to FIGS. 4 to 6. In this method for manufacturing, the conductors 22 are formed on the surface of the flexible board 20 which is a planar member, and the flexible board 20 on which the conductors 22 are formed is deformed into a column shape such that the conductors 22 intersect with the circumference direction of the flexible board 20 or extend in the direction perpendicular to the circumference direction. Further, the electrode-side end face 20c of the flexible board 20 deformed into a column shape is cut to expose the conductors 22 located at the end portion on the first end side of the conductors 22, and electrode-side end face 20c on which the wires 22 are exposed is polished such that the conductors 22 come to be flush with the electrode-side end face 20c.

### Manufacturing Steps 1 to 3

FIG. 4 is a diagram illustrating a process of manufacturing the transmitter device 1 including the lens 41 of the first embodiment. In Step 1, in order to form the conductors 22, a circuit layer (e.g., Cu layer) 23a conforming to a wiring shape is patterned on the surface of the FPC 20 by means of a photofabrication technique (item “1” in FIG. 4). The present embodiment uses the LCP 21 as the FCP 20. In Step 2, the insulating layer 24 is deposited at a predetermined thickness (item “2” in FIG. 4). In the present embodiment, a layer of parylene (polyparaxylene) was formed at a thickness of 5 μm as the insulating layer 24. In Step 3, reshaping is performed on the fabricated FPC 20 (item “3” in FIG. 4). The item 3 of FIG. 4 is a schematic diagram illustrating an internal structure of the transmitter device 1 before being cut.

### Reshaping step I to IX

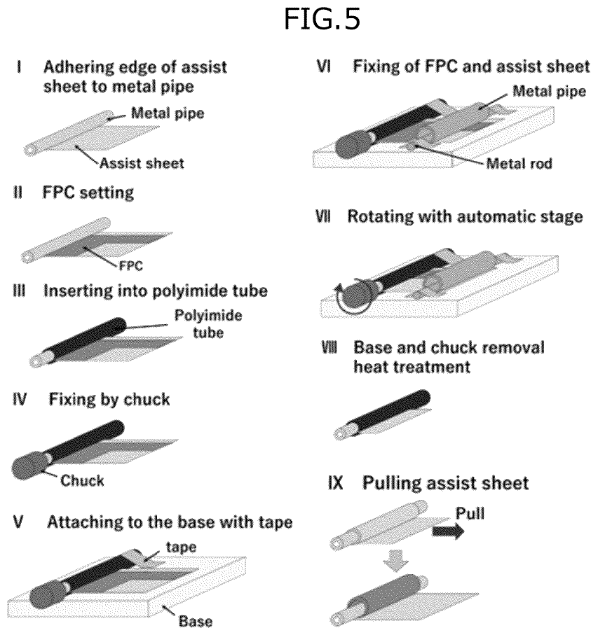

FIG. 5 is a diagram illustrating a reshaping process of reshaping the FPC 20. In this example, the FPC 20 is reshaped into a circle. First, a winding jig is fabricated by biding an assist sheet (e.g., LCP) for winding and a metallic pipe together with an adhesive (FIG. 5I). Then the FPC 20 is placed between the assist sheet and the metallic pipe (FIG. 5II). Next, a resin-made tube (e.g., polyimide tube) with a notch is wrapped over the jig (FIG. 5III), and then fixed to an automatic stage (FIG. 5IV). Then, after only the resin tube is fixed to a base (FIG. 5V), the assist sheet is fixed by the metal pipe and a metal rod to pull the assist sheet (FIG. 5VI). Then, after the FPC 20 is wrapped by rotating the automatic stage (FIG. 5VII), the fixation is released and heat treatment is performed at a predetermined temperature for a predetermined time (e.g., at 150° C. for 30 minutes) (FIG. 5VIII). Finally, the resin tube is removed and the reshaped FPC 20 is taken out by widening the assist sheet (FIG. 5IX). By undergoing the reshaping process, the FPC 20 is deformed into a column shape such that the conductors 22 extend in a direction intersecting with the circumferential direction of the flexible board 20.

By wrapping the FPC 20 inside the resin tube serving as the fixing jig, this method avoids loosening of the assist sheet generated when the FPC 20 is fixed after being wrapped.

In the above reshaping process of this example, the flexible board 20 in a column shape is wrapped around the metal pipe serving as a core material and then the core material is removed. Alternatively, the core material may be etched away.

### Manufacturing Step 4

Referring back to FIG. 4, in Step 4, the reshaped FPC 20 is bonded to the supporting body 10 with an adhesive (item “4” in FIG. 4). In the present embodiment, AlON Alpha A (registered trademark, Daiichi Sankyo Co., Ltd.) was used as the adhesive, and a ceramic tube was used as the supporting body 10. The item “3” of FIG. 4 is a schematic diagram illustrating the internal structure of the transmitter device 1 before the cutting.

If the flexible board is sufficiently thin or flexible, the process may proceed to the next biding process, omitting the reshaping process.

### Binding Processes I to IV

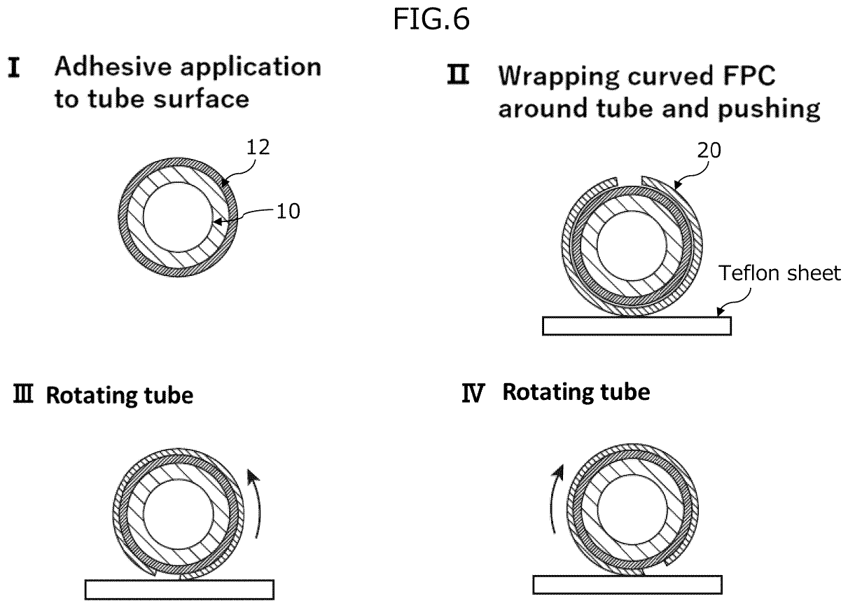

FIG. 6 is a diagram illustrating a biding process of biding the FPC 20 to the supporting body 10. First, an adhesive is applied to the supporting body 10 with a brush, and then fixed to the automatic stage (not illustrated) (FIG. 6I). After that, the supporting body 10 is pushed into the FPC 20 (FIG. 6II). The overall of the FPC 20 from the first end to the second end along the left-right direction is bounded to the supporting body 10 by rotating the supporting body 10 using the automatic stage while the supporting body 10 is pressed against the base (e.g., Teflon® sheet) (FIGS. 6II, 6III and 6IV). The pressing was performed by pushing the metal pipe into the through-path 11 of the supporting body 10, avoiding the direct contact with the FPC 20, and rotating the supporting body 10 along with the metal pipe.

### Manufacturing Steps 5 to 9

After the biding step, returning to FIG. 4, in Step 5, the FPC 20 is cut at a predetermined length (e.g., 0.5 mm) together with the supporting body 10 with a dicer (e.g., DISCO DAD522) to expose the electrode-side end face 20c to form the recording electrode 23 (item “5” in FIG. 4). Step 5 exposes the conductor 22 located at the first ends of the conductors 22, which is also at the end portion.

In Step 6, the lens 41 is fit and fixed into the end portion (front end portion) on the inner side of the flexible board 20 (item “6” in FIG. 4). The supporting body 10 and the lens 41 are bonded together with the adhesive 12.

In Step 7, the entire surface of the FPC 20 (i.e., outer side of the insulating layer 24) is coated with the adhesive 12 (item “7” in FIG. 4). In Step 8, the conductors 22 are exposed, and the electrode-side end face 20c in which the lens 41 is fitted is polished such that the conductors 22 and the lens 41 come to be flush with the electrode-side end face 20c. Since uneven burrs are formed on the metallic layer (Cu layer) after the cutting in Step 5, the burrs are removed by etching in order to make the areas of the recording electrodes uniform (item “8” in FIG. 4). In the final Step 9, metal plating at a predetermined thickness (1 μm thickness of Ni, 1 μm thickness of Au; plating layers 23b, 23c) is performed on the conductors 22 located on the electrode-side end surface 20c (item “9” in FIG. 4). Through Steps 1-9 of the manufacturing process, the transmitter device 1 including the recording electrodes 23 and the lens 41 and formed of the flexible board 20 illustrated in FIGS. 1-3 is completed.

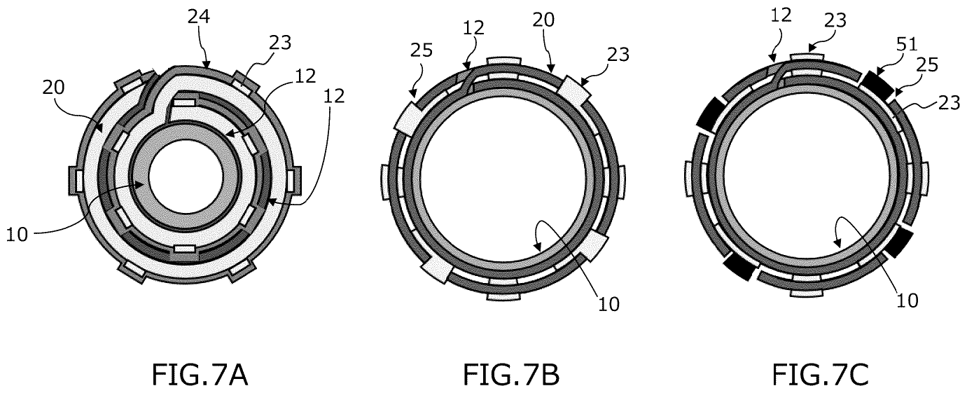

The transmitter device 1 may implement a small-sized electronic component and/or an optical component (mounting component) 51 as required on the surface of the FCP 20. FIG. 7C illustrates an example in which the mounting component 51 is mounted on the FPC 20. FIG. 7C illustrates an example in which FPC 20 is wrapped in multiple times (multiple plies), and alternatively, the mounting component 51 can be mounted on the surface of the FPC 20 even in an embodiment in which FPC 20 is wrapped a single time.

### 3. Action and Effect

(1) In the above-described transmitter device 1, the conductors 22 are flush with the electrode-side end surface 20c. In the structure that obtains a signal from a measuring target by bring the electrode-side end face 20c close to or in contact with the measuring target, the transmitter device 1 can obtain more accurate signal than a device that includes conductors 22 on its long-axial face because the observation site matches the measuring site in the transmitter device 1.
(2) In addition, the above method for manufacturing the transmitter device 1 is simple and has a small number of steps, so that the time required for manufacturing can be shortened and consequently, the productivity can be enhanced.
(3) The member formed or deformed in a column shape makes it possible to non-planarly mount the conductors 22 on a face of the transmitter device 1.
(4) By using the flexible board 20 as a member, a shape can be easily produced, so that the transmitter device 1 compact in size can be manufactured. In addition, since a non-planar shape can be produced by planar processing, the operation becomes simple.
(5) Since the lens 41 is also made flush with the electrode-side end surface 20c along with the conductors 22, the observation face of the lens 41 matches the measurement face of the conductors 22, which enables more accurate measurement.

### A-1. Modification of First Embodiment

The first embodiment assumes the FPC 20 is a single ply, but the FPC 20 may be wrapped multiple times (in multiple plies). FIGS. 7A-7C are schematic diagrams of a transmitter device 1 in which the FPC 20 is wrapped multiple times (i.e., in multiple plies). In the transmitter device 1 of the first modification, the recording electrodes 23 are formed on an end face of multiple flexible boards 20 wrapped multiple times (i.e., in multiple plies).

### 1. Configuration

FIG. 7A illustrates a transmitter device 1 in which multiple layers are formed by the FPC 20.

The FPC 20 may further be provided with a through-hole 25. FIG. 7B illustrates a transmitter device 1 formed into multi-layer structure by the FPC 20 including the through-hole 25. The through-hole 25 is for interconnecting the conductors 22 on the flexible board 20 wrapped in multiple plies. The interconnection is accomplished by connecting the conductors 22 of the upper and lower layers with wiring via the through-hole 25. For example, a portion where the conductor 22 on the lower layer and the conductor 22 on the upper layer are integrated in FIG. 7B is a portion connected by wiring via the through-hole 25.

FIG. 7C shows a transmitter device 1 in which a multi-layer structure is formed with a FPC 20 where a small electronic component and/or an optical component 51 is mounted. The through-hole 25 in this example is for avoiding overlap between the small-sized electronic component and/or the optical component 51 mounted on the surface of the flexible board 20 and the flexible board 20. In other words, the through-hole 25 is for allowing the mounting component 51 on an inside FPC 20 among the FPC 20 wrapped in multiple plies to protrude upward through the through-hole 25 on the outside FPC 20.

### 2. Actions and Effects

By forming FPC 20 into a multi-layer structure, the number of recording electrodes 23 can be increased. Furthermore, by providing through-holes 25, the transmitter device 1 can be made compact in size even if the FPC 20 is formed into multiple layers. Furthermore, in a structure in which the FPC 20 with the mounting component 51 on the surface thereon is formed into multi-layer structure, providing the through-holes 25 makes it possible to avoid generation of a gap between an inside FPC 20 and an outside FPC 20 which generation is caused by the FPC 20 covering on the mounting component 51. As described above, by providing the through-hole 25, the degree of freedom in structure and arrangement is enhanced, and the functions of the transmitter device 1 can be increased and enhanced.

In the first embodiment and the modification thereof, the transmitter device 1 includes the supporting body 10, but the supporting body 10 may be omitted. This means that the above description relates to an example that wraps the FPC 20 around the supporting body 10 and then binds the FPC 20 and the supporting body 10, but alternatively the column shape may be formed only by the FPC 20. The method for manufacturing a transmitter device 1 that forms the column shape only by the FPC 20 and does not include the lens 41 includes the manufacturing steps 1-3 (including reshaping steps I to IX in FIG. 5 related to the manufacturing step 3) of FIG. 4 of the first embodiment, but does not perform the manufacturing steps 4-9 (including adhering steps I to IV of FIG, 6) of FIG. 4. If the column shape is formed only by the FPC 20, the transmitter device 1 illustrated in FIG. 3 does not include the supporting body 10 and the adhesive 12 between the supporting body 10 and the supporting body 10 and the FPC 20.

Furthermore, the lens 41 may be fitted into the inside of the through-path 11 of the FPC 20. The method for manufacturing a transmitter device 1 that forms the column shape only by the FPC 20 and includes the lens 41 does not use the adhesive 12 applied between the supporting body 10 and the FPC 20 in the manufacturing steps 5-9 of FIG. 4, and binds the lens 41 to FPC 20 with the adhesive 12.

Also the configuration that forms the column shape only by the FPC 20 can wrap the FCP 20 multiple times, form the through-holes 25 on the FCP 20, and mount the mounting component 51 on the FCP 20. In this case, the configurations of FIGS. 7A to 7C each do not include the supporting body 10.

### B. Second Embodiment

### 1. Configuration

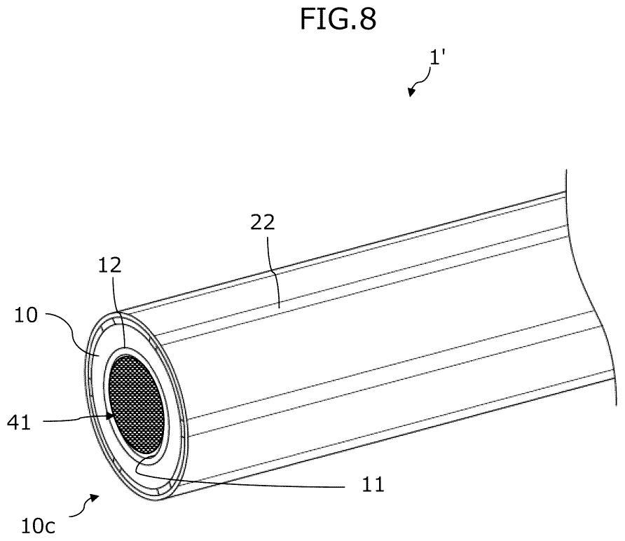

Description will now be made in relation to a transmitter device 1′ according to a second embodiment with reference to FIGS. 8 to 10. The second embodiment is different from the first embodiment in the point that the flexible board 20 is not used. Hereinafter, description mainly focuses on the difference. In the description of the second embodiment, like reference signs designates the same or substantially same elements as the first embodiment, so repetitious description is omitted.

FIG. 8 is a schematic diagram illustrating a configuration of a transmitter device 1′ formed of the supporting body 10. The supporting body 10 is a member (material) formed in a column shape such as a cylindrical shape or a prism shape. On the surface of the supporting body 10, the conductors 22 are formed so as to extend in a direction intersecting with the circumference direction of the supporting body 10. The adhesive 12 is applied to an end portion of the supporting body 10, the electrode-side end face 10c to which the adhesive 12 is applied is polished, and the conductors 22 are flush with the electrode-side end face 10c.

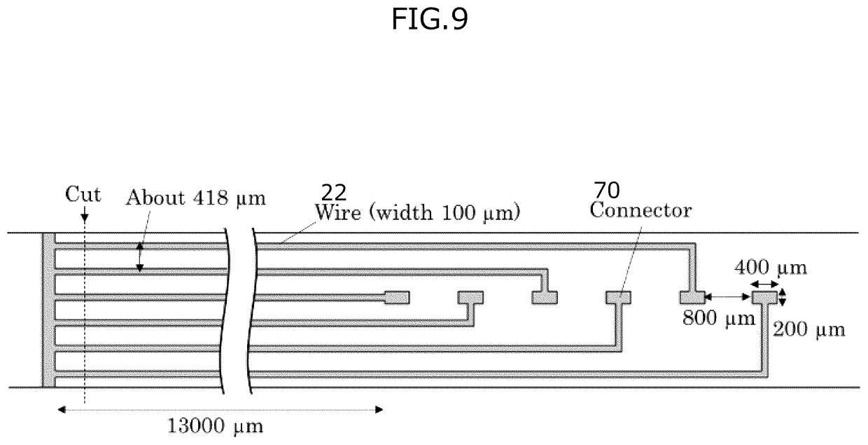

FIG. 9 is a schematic diagram of a wiring pattern formed on the surface of the supporting body 10. In the present embodiment, wiring as the conductors 22 is made of, for example, Au, and six conductors are arranged at equal intervals in the circumferential direction of the supporting body 10.

Returning to FIG. 8, the supporting body 10 has a through-path 11 that penetrates the electrode-side end face 10c and a second end surface on the opposite side of the electrode-side end face 10c. A lens 41 may be fitted into the electrode-side end face 10c in the through-path 11.

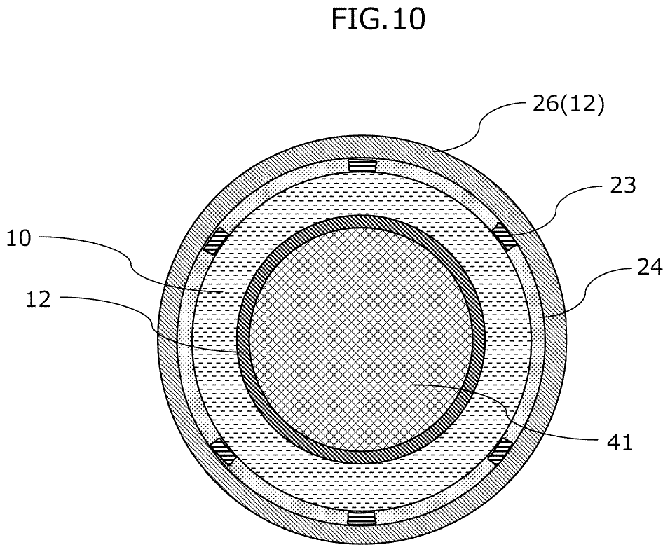

FIG. 10 is a schematic diagram illustrating a front end face of the transmitter device 1′. The electrode-side end face 10c includes, sequentially in the outward direction from the center of the through-path 11, the lens 41, the adhesive 12, the supporting body 10, the adhesive 12, the recording electrode 23 formed at the end faces of the conductors 22, an insulating layer 24 covering the supporting body 10 and the conductor 22 (the recording electrode 23 in FIG. 1), and an protective layer 26 (adhesive 12). The recording electrodes 23 are provided at equal intervals, and the angle between the electrodes may be 60 degrees, for example.

### 2. Method for Manufacturing

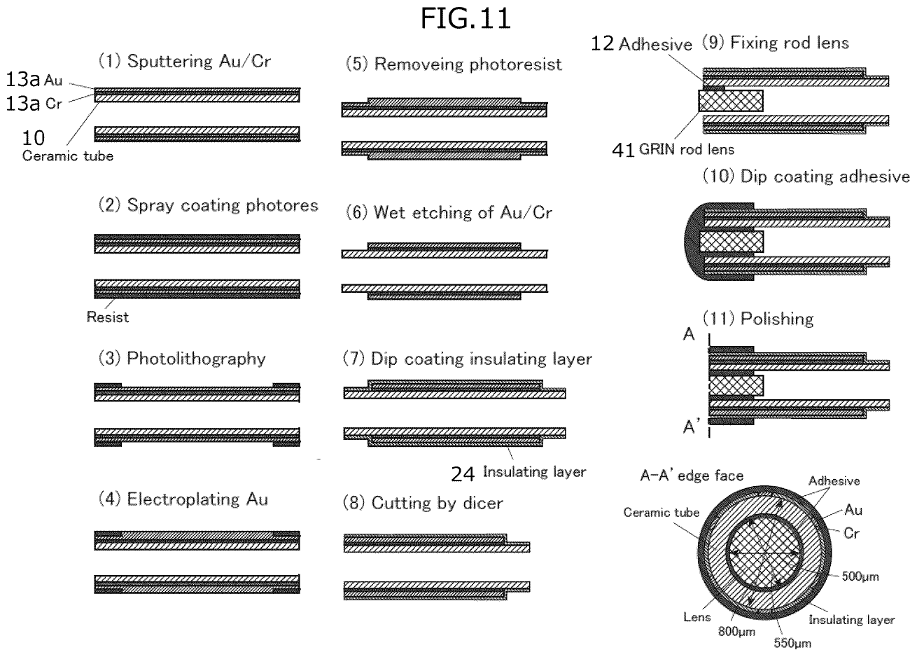

Next, a method of manufacturing the transmitter device 1′ formed of the supporting body 10 will now be described with reference to FIG. 11. The manufacturing method forms the conductors 22 on the surface of the supporting body 10 which is a member formed in a column shape such as a cylindrical shape or a prism shape such that the conductors 22 extend in a direction intersecting with the circumferential direction of the member, applies the adhesive 12 to an end portion of the supporting body 10 on which portion the conductors 22 are formed, and polishes the electrode-side end face 10c applied with the adhesive 12 to make the conductors 22 flush with the electrode-side end face 10c. Furthermore, with respect to the supporting body 10 including a through-path 11 that penetrates the electrode-side end face 10c and a second end surface on the opposite side of the electrode-side end face 10c, the method inserts the lens 41 into at end portion in the through-path 11 of the supporting body 10 on which the conductors 22 are formed, applies the adhesive 12 to the end portion of the supporting body 10 fitted with the lens 41, and polishes the electrode-side end face 10c applied with the adhesive 12 to thereby make the recording electrode 23 and the lens 41 flush with the electrode-side end face 10c.

FIG. 11 is a diagram illustrating a process for manufacturing the transmitter device 1′ formed on the supporting body 10. In FIG. 11, the process is described with reference to a cross-sectional view of the supporting body 10 in the up-down direction. In the first step (1), firstly, a metallic layer (circuitry layer 13a, Cr, Au) is formed on the surface of the supporting body 10 (e.g., ceramic tube) by sputtering. In the second step (2), a positive resist (OFPR 34cP, Tokyo Ohka Kogyo Co., Ltd.) is uniformly applied on the metallic layer while the supporting body 10 is being rotated using a spray coater and an automatic stage, and in the third step (3), patterning is carried out by point-exposing a wiring portion and a connecting portion using a non-planar exposure device and then carrying out development. In a fourth step (4), Au is electroplated on the formed wiring pattern at a predetermined thickness (about 2.5 μm).

In a fifth step (5), the photoresist is removed, and in a sixth step (6), the seed layer is removed by etching, and in a seventh step (7), the photosensitive polyimide (PW-1200, Toray Co., Ltd.) is applied to form the insulating layer 24 at a thickness of 10 μm by dip coating. In the ensuing eighth step (8), wiring is cut with a dicer at the desired embedding length.

In the ninth step (9), the cylindrical GRIN lenses 41 provisionally fixed to the tip portion of the cut tube using EPO-TEK MED-302-3M (Epoxy Technology. Inc) as a thermosetting adhesive 12 that is biocompatible and excellent in water resistance. At this time, in order to suppress the polishing amount of the GRIN lens within an allowable range of 100 μm, the amount of protrusion from the tube is set to be less than 100 μm. In the subsequent tenth step (10), the thermosetting adhesive 12 is dip-coated to fill the gap between the lenses 41 and the supporting body 10 and a protective layer 26 is formed on the outer side of the supporting body 10 to prevent the insulating layer 24 from peeling off during the polishing. In the last eleventh step (11), wires are exposed on the electrode-side end face 10c by polishing the supporting body 10 with the conductors 22 and the GRIN lens 41 at the same time using SFP-70D2 (Seiko Giken), which is a polishing machine for optical fibers, and thereby an electrode is fabricated. The above process successfully arranges the end surface of the lens and the electrode face on the same plane (i.e., the end surface of the lens is flush with the electrode face). Referring to a planar polishing step of an optical fiber, the polishing process is performed in the order of, for example, removing the adhesive (polishing film: GA07), primary polishing (polishing film: DR07-9U), secondary polishing (polishing film: DR07-1U), and finish polishing (polishing film: XF07). The first to eleventh steps of the manufacturing process complete to manufacture the transmitter device 1′ including the recording electrode 23 and the lens 41 and being formed of the supporting body 10 as illustrated in FIGS. 8 to 10 and the lower right of FIG. 11.

### 3. Action and Effect

The transmitter device 1′ of the second embodiment can attain all the effects of the first embodiment except for the effects of the flexible board 20. Here, effects peculiar to the second embodiment will now be described. By printing the conductors 22 directly on the supporting body 10 serving as a member, the number of steps can be reduced as compared to the method for manufacturing the transmitter device 1 made of the FPC 20.

### C. Third Embodiment

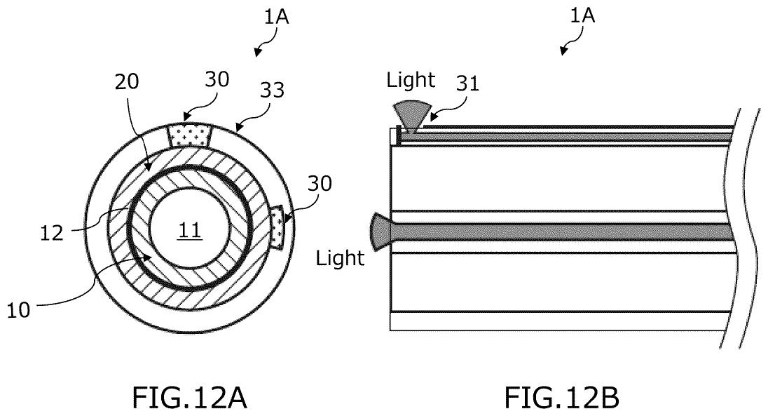

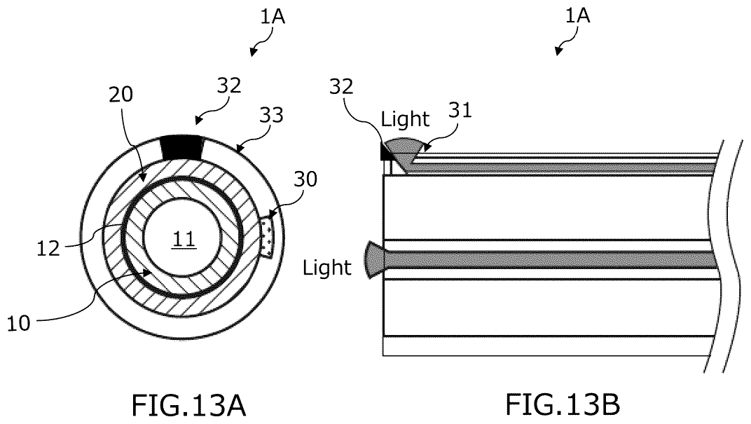

The transmitter devices 1, 1′according to a first embodiment and the modification thereof and the second embodiment may include the optical waveguides 30 in place of the conductors (conductor tracks, wires) 22, or may include one or more optical waveguides 30 in addition to the conductors 22. Referring to FIGS. 12 and 13, the configuration of an optical waveguide 30 will now be briefly described. In the description of the third embodiment, like reference signs designates the same or substantially same elements as the first and second embodiments, so repetitious description is omitted.

FIGS. 12A and 12B are schematic diagrams illustrating a configuration of the transmitter device 1A having an optical waveguides 30 in place of wires 22. The transmitter device 1A illustrated in FIG. 12 is different from the transmitter device 1 of the first embodiment in the point that the transmitter device 1A forms the optical waveguides 30 on the surface of the FPC 20 in place of the conductors 22.

FIG. 12A illustrates the front end face of the transmitter device 1A. The transmitter device 1A includes, sequentially in the outward direction from the through-path 11, the supporting body 10, the adhesive 12, the FPC 20, the waveguides 30, and a coating layer 33. Each optical waveguide 30 formed on the transmitter device 1A of FIG. 12A includes an optical window 31 to be described below and therefore penetrates the coating layer 33. Each optical waveguide 30 is composed of the FPC 20, a core through which light propagates between the FPC 20 and the coating layer 33 that coats the FPC 20, and a cladding that encloses the circumference of the core not to dissipate the light. The optical waveguide can be formed using, for example, a photoresist having a high light transmittance for the core and using a metallic mirror that reflects light for the cladding. FIG. 12B illustrates the internal configuration of a side face (a face extends in front-rear direction) of the transmitter device 1A and corresponds to FIG. 12A. The optical waveguides 30 extend in the front-rear direction of the transmitter device 1A. As shown in FIG. 12B, the optical window 31 formed by removing the coating layer 33 is provided at a front end portion of the transmitter device 1A. The optical windows 31 are provided on the front end face or the front portion of the side face of the transmitter device 1A. Although the lens 41 is not fitted in FIG. 12, the lens 41 may be inserted into the front end portion of the through-path 11 so that the lens 41 may be provided on the front end face.

As shown in FIG. 12B, the light enters from second end side on the opposite side (rear end side) of the front end face of the transmitter device 1A, proceeds to the front end face, and is emitted from the optical window 31. If the optical window 31 is opened forward, the light is emitted forward, and if the optical window 31 is opened to the side, the light is emitted to the side (in this example upward). By providing the optical waveguides 30 and the optical windows 31, light can be emitted in the axial direction or the circumference direction.

FIG. 13A illustrates a transmitter device 1A that further provides a mirror 32 to one of the optical waveguides 30. As shown in FIG. 13B, the light entered from the rear end face is reflected on the mirror 32 provided to the optical window 31, and is emitted from the optical window 31. The mirror 32 can bend the light path of the waveguide to the circumference direction (i.e., toward the side face of the transmitter device 1A) of the FPC 20, and also can change the direction in which light is emitted depending on the angle of the mirror 32. In this example, a mirror is assumed to be inclined by 45°.

FIG. 12 and FIG. 13 illustrate examples each having optical waveguides 30 in place of the conductors 22, but alternatively, each transmitter device 1A may include one or more optical waveguides 30 in addition to the conductors 22. In this alternative, the conductors 22 and the optical waveguides 30 are arranged side by side.

FIG. 12 and FIG. 13 illustrate examples of the transmitter devices 1A each formed of the FPC 20, but alternatively the optical waveguides 30 may be formed on the supporting body 10. This means that, the optical waveguides 30 may be formed on the surface of the supporting body 10 of the transmitter device 1′of the second embodiment in place of or in addition to the conductors 22.

If the optical waveguides 30 of the third embodiment is applied to the modification of the first embodiment, the through-hole 25 is for interconnecting the optical waveguides 30 on the flexible board 20 wrapped in multiple plies. The interconnection is accomplished by connecting the optical waveguides 30 of the upper and lower layers via an optical fiber.

The transmitter device 1A of third embodiment brings the above effects, and additionally obtains all the effects of the first and second embodiment.

### D. Applied Example

The above-described transmitter devices 1, 1′, and 1A can have additional functions to the electric measurement and the optical measurement by combining another device through the through-path 11. Hereinafter, description will now be made in relation to an application example of the transmitter device 1, and the transmitter device 1′ and the transmitter device 1A are also applicable to an application example.

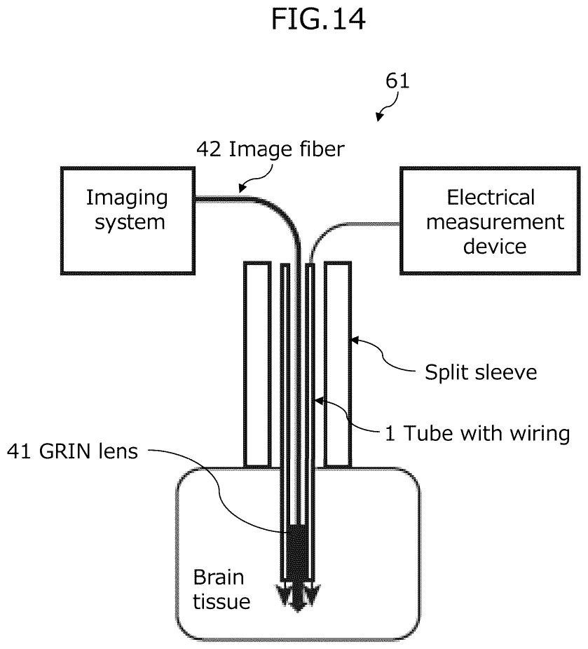

FIG. 14 is a diagram illustrating an endoscope apparatus 61 with the transmitter device 1. An endoscope 42 is inserted into the through-path 11 of the transmitter device 1 and the endoscope 42 and the transmitter device 1 are used in combination. As shown by the arrows in FIG. 14, the observation direction (one thick arrow) of the transmitter device 1 inserted into the brain tissue matches the electric measurement direction (two thin arrows). When being combined with the endoscope 42, the transmitter device 1 can observe the site of neuron where the neurotransmission has occurred, and at the same time, can measure the potential with the recording electrodes 23 (not illustrated) arranged on the outer side. This enables analysis at high temporal resolution and high spatial resolution. The transmitter device 1 used in combination with the endoscope 42 makes it possible utilize a technique of calcium imaging. This technique is intended for calcium-ions that increase its concentration in a neuron during neurotransmission, and images a neuron where the neurotransmission occurred to fluoresce at the endoscopic observation unit. This imaging has been applied to cultured brain tissue using a microscope, but the present ultra-fine fluorescent endoscope can carry out the in-vivo imaging on an active brain.

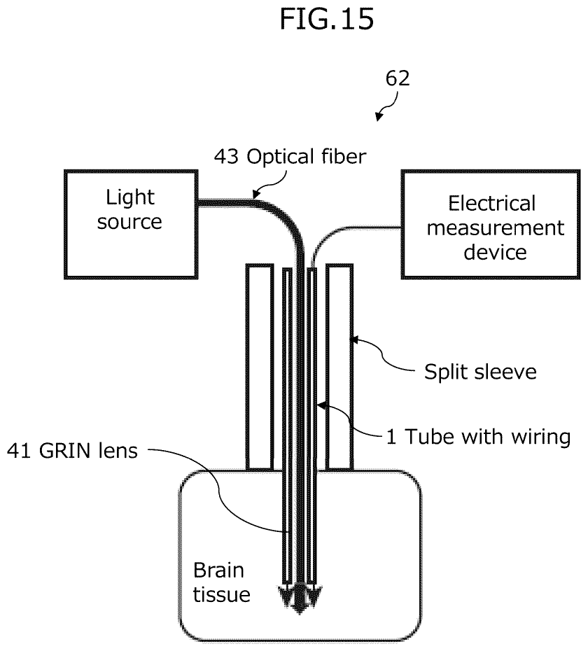

FIG. 15 is a diagram illustrating an optical fiber apparatus 62 with a transmitter device 1. An optical fiber 43 is inserted into the through-path 11 of the transmitter device 1 and the optical fiber 43 and the transmitter device 1 are used in combination. As shown by the arrows in FIG. 15, the simulation direction (one thick arrow) of the transmitter device 1 inserted into the brain tissue matches the electric measurement direction (two thin arrows). By using this combination with the optical fiber 43 being inserted in brain tissue, the measurement is carried out while optical stimulation is giving to the brain tissue. The transmitter device 1 used in combination with the optical fiber 43 makes it possible to utilize a technique of optogenetics. In contrast to related electrical stimulation, which stimulates tissues except for target tissue, the technique optogenetics can stimulate only the target tissue, enabling active measurement of a specific site.

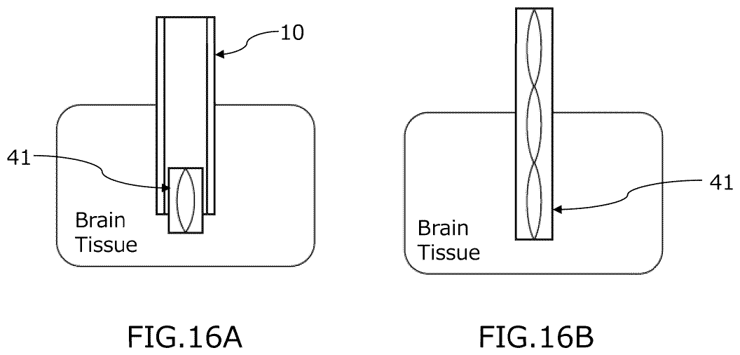

Here, description will now be made in relation to a GRIN lens 41, which is related to a device to be combined with the transmitter device in an application example. A GRIN lens 41 obtains an effect of bending light by parabolically distributing a refractive index from the center axis to the outer circumference of the glass, and has a property that, when being polished to be relatively shorter than the entire length, only deviates the focal position, which can be corrected by adjusting the relative position between the imaging fiber and the GRIN lens 41. For deep brain imaging, it is desirable to use a short GRIN lens with a small aberration. FIG. 16A and FIG. 16B are schematic diagrams illustrating GRIN lenses 41 with small aberrations.

FIG. 16A illustrates a GRIN lens having a 0.5 pitch. A 0.5 pitch means that the period of light cycle is half a cycle. In this example, since light bends once from image formation at one end of the lens 41 to the next image formation at the other end of the lens 41 (which means half the cycle), the light propagating through the lens 41 is less affected by deviation of a wavelength and deviation of a refractive index of a peripheral portion. In other words, the 0.5-pitch GRIN lenses 41 is less likely to generate an aberration. On the other hand, FIG. 16B illustrates a GRIN lens having a 1.5 pitch. In this example, since the light refraction occurs three times, the GRIN lenses 41 is likely to generate aberration.

A 0.5-pitch GRIN lens is suitable for imaging and is preferably used in combination with an endoscope. A lens having a pitch of an integer (0,1,2,3 . . . )+0.5 is optically connected to the endoscope apparatus via a cable extending in the through-path 11.

On the other hand, a GRIN lens having a pitch of 0.25 can emit parallel light and is suitable for being used in combination with an optical fiber. A 0.25 pitch means that the period of light cycle is one-fourth a cycle. A lens having a pitch of an integer (0,1,2,3 . . . )+0.25 is optically connected to an optical fiber apparatus via a cable extending in the through-path 11.

### E. Miscellaneous

The materials and members described in the above embodiments are not limited thereto. Further, the embodiments are applied to a neural electrode as a specific example, but the application of the embodiments is not limited to this. For example, the transmitter device 1 may alternatively be used as a probe for diagnosing degradation of a building. In addition, the application example is not limited to a combination with an endoscope or an optical fiber, and the transmitter device 1 may alternatively be used as a probe that is inserted into a body or a complex device and performs measurement or activation at the tip portion of the probe. In this alternative, by adjusting the inner diameter of the through-path 11 according to the size of the target to be measured, the transmitter device 1 can be combined with another device in the form of, for example, the thin cord or a thin rod.

According to the present disclosure, it is possible to manufacture a transmitter device that can measure a site being observed in a simple procedure.

Throughout the descriptions, the indefinite article “a” or “an”, or adjective “one” does not exclude a plurality.

All examples and conditional language recited herein are intended for the pedagogical purposes of aiding the reader in understanding the disclosure and the concepts contributed by the inventor to further the art, and are not to be construed limitations to such specifically recited examples and conditions, nor does the organization of such examples in the specification relate to a showing of the superiority and inferiority of the disclosure. Although one or more embodiments of the present disclosures have been described in detail, it should be understood that the various changes, substitutions, and alterations could be made hereto without departing from the spirit and scope of the disclosure.

What is claimed is:

1. A method for manufacturing a transmitter device comprising:
forming a transmitter on a member in a form of a column shape or deformed into a column shape;
providing a light guide to an end portion of the member; and
carrying out a process on an end face of the member to expose the transmitter and the light guide such that the transmitter and the light guide are flush with the end face of the member.

2. The method according to claim 1, further comprising:
forming the transmitter on a surface of a flexible board serving as a planer member;
deforming the flexible board with the transmitter into a column shape such that the transmitter extends in a direction intersecting with a circumferential direction of the member;
cutting the end face of the flexible board deformed into the column shape to expose a first end of the transmitter, the first end being positioned at the end portion;
fitting the light guide into the end portion at an inner side of the flexible board deformed into the column shape; and
polishing the end face such that the transmitter and the light guide are flush with the end face.

3. The method according to claim 2, further comprising:
after forming the transmitter on the surface of the flexible board, mounting a small-sized electronic component and/or an optical component on the surface of the flexible board.

4. The method according to claim 2, further comprising:
wrapping the flexible board around a core material; and
removing or etching away the core material.

5. The method according to claim 3, further comprising wrapping the flexible board in multiple plies.

6. The method according to claim 5, further comprising:
forming a through-hole on the flexible board, the through-hole connecting the transmitter on the flexible board wrapped in multiple plies to each other, the through-hole avoiding overlap between the flexible board and the small-sized electronic component and/or the optical component mounted on the surface of the flexible board.

7. The method according to claim 1, further comprising:
fitting the light guide into the end portion of the member in the column shape;
making the transmitter and the light guide flush with the end face by polishing the end face of the column member fit with the light guide.

8. The method according to claim 1, further comprising:
forming the transmitter on a surface of material, serving as the member formed into a cylindrical or prism shape as a column shape, such that the transmitter extends in a direction intersecting with a circumference direction of the member;
fitting the light guide into the end portion of the material with the transmitter formed in the forming,
applying an adhesive to the end portion; and
making the transmitter flush with the end face by polishing the end face applied with the adhesive.

9. The method according to claim 1, further comprising:
fitting the light guide into the end portion of a through-path of a material forming the transmitter, the through-path penetrating the material from the end face and another face on an opposite end of the end face;
applying an adhesive to the end portion of the material fit with the light guide; and
making the transmitter flush with the end face by polishing the end face applied with the adhesive.

10. A transmitter device comprising:
a member deformed into a column shape, the member being a flexible board wrapped around a circumference face of the column shape in multiple plies; and
a transmitter provided on the member, and
a light guide provided at an end portion of the member, wherein
the transmitter and the light guide are configured to be flush with a plurality of end faces of the flexible board wrapped in multiple plies.

11. The transmitter device according to claim 10, wherein
the member deformed into the column shape comprises a through-path penetrating the member from the end face and another face on an opposite end of the end face.

12. The transmitter device according to claim 10, wherein
the flexible board comprises a through-hole connecting the transmitter on the flexible board wrapped in multiple plies to each other, the through-hole avoiding overlap between the flexible board and a small-sized electronic component and/or an optical component mounted on the flexible board.

13. The transmitter device according to claim 11, wherein
the light guide is a lens having a pitch of an integer (0,1,2,3 . . . )+0.5, and is optically connected to an endoscopic device via a cable extending through the through-path.

14. The transmitter device according to claim 11,
the light guide is a lens having a pitch of an integer (0,1,2,3 . . . )+0.25, and is optically connected to an optical fiber device via a cable extending through the through-path.
